SEQUENCE LISTING (5 sequences)

SEQ ID NO: 1 - DNA, 59 bp, Artificial Sequence
  5′ primer 1sense
gggaattccatatggattgcggcggcggcggcagcaaaggcgaaacaactactgaagct

SEQ ID NO: 2 - DNA, 34 bp, Artificial Sequence
  3′ primerantisense
gagctcgagttcagttaccgtaaaggtcttagtc

SEQ ID NO: 3 - DNA, 15 bp, Artificial Sequence
  Primer
ggcggcggcggcagc

SEQ ID NO: 4 - protein, 5 aa, Artificial Sequence
  Peptide linker
DDDDK

SEQ ID NO: 5 - protein, 55 aa, Artificial Sequence
  Protein G partial sequence
TYKLVINGKTLKGETTTKAVDAETAEKAFKQYANDNGVDGVWTYDDATKTFTVTE

(19) United States
(12) Patent Application Publication
(10) Pub. No.: US 2010/0203653 A1
(43) Pub. Date: Aug. 12, 2010

(54) Protein G-Oligonucleotide Conjugate

(76) Inventors: Bong Hyun Chung, Daejeon (KR); Yong Won Jung, Daejeon (KR); Jeong Min Lee, Daejeon (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/665,397

(22) PCT Filed: May 16, 2008
(86) PCT No.: PCT/KR08/02739

§ 371 Date: Dec. 18, 2009

(30) Foreign Application Priority Data
Jun. 18, 2007 (KR) 10-2007-0059643

(51) Int. Cl. G01N 33/543; C07K 14/00

(52) U.S. Cl. 436/518; 530/409

(57) ABSTRACT

The present invention relates to a protein G conjugate, which is prepared by linking an N-terminal cysteine-tagged protein G variant with an oligonucleotide via a linker. The conjugate binds in a directional manner on the surface of a biochip and biosensor, thereby providing a biochip and biosensor having improved antibody immobilization ability.

### TECHNICAL FIELD

The present invention relates to a protein G conjugate (gA-G) which is prepared by linking an N-terminal cysteine-tagged protein G variant with an oligonucleotide using a linker, a method for preparing the same, and a biochip and a biosensor fabricated by using the conjugate.

### BACKGROUND ART

Antibodies have been widely used in medical studies concerning diagnosis and treatment of diseases as well as in biological analyses, because of their property of specifically binding to an antigen (Curr. Opin. Biotechnol. 12 (2001) 65-69, Curr. Opin. Chem. Biol. 5 (2001) 40-45). Recently, as an immunoassay, immunosensors have been developed, which require the immobilization of an antibody on a solid support to measure changes in current, resistance and mass, optical properties or the like (Affinity Biosensors. vol. 7: Techniques and protocols). Among them, a surface plasmon resonance-based immunosensor making use of optical properties has been commercialized. The surface plasmon resonance-based biosensor provides qualitative information (whether two molecules specifically bind to each other) and quantitative information (reaction kinetics and equilibrium constants), and also performs sensing in real time without the use of labeling, thus being particularly useful for measuring antigen-antibody binding (J. Mol. Recognit. 1999, 12, 390-408).

In the immunosensor, it is very important that antibodies are selectively and stably immobilized on a solid support. The techniques for immobilizing antibodies are classified into two categories, physical immobilization and chemical immobilization. The physical immobilization techniques (Trends Anal. Chem. 2000 19, 530-540) have been minimally used because they cause denaturation of the protein, and the results are less reproducible. In contrast, the chemical immobilization techniques (Langumur, 1997, 13, 6485-6490) have been widely used because they show good reproducibility and a wide range of applications, due to their feature of allowing secure binding of proteins through covalent bonding. However, when immobilization of antibodies is performed using a chemical immobilization technique, the antibodies, being asymmetric macromolecules, often lose their orientation and activity to bind to antigens (Analyst 121, 29R-32R).

In an attempt to enhance the ability of antibodies to bind to antigens, a support may be used before the antibodies are linked to a solid substrate, and a technology of using protein G as the support is known. However, there is a problem that this protein G itself also loses orientation and its ability to bind to an antibody when bound to the support.

Accordingly, in order to solve such problem, a variety of methods have been suggested. For example, Streptococcal protein G is treated with 2-iminothiolane to thiolate the amino acid group of a protein, and then the thiolated Streptococcal protein G is immobilized on the surface. However, this method is directed to thiolating the amino groups of amino acids having an amino group (Arg, Asn, Gln, Lys), instead of thiolating any specific site, and thus the method results in low specificity and requires additional purification processes after chemical treatments (Biosensors and Bioelectronics, 2005, 21, 103-110).

A DNA-directed immobilization method has been used for immobilization of protein. The DNA surface is known to be stable, and known to be easily prepared, as compared to a protein chip. For the protein immobilization, the following factors have to be considered, such as storage for a long period of time, immobilization of unstable protein, or protein storage under unstable conditions. The DNA-directed antibody immobilization methods have also been reported, for example, an immobilization method of biotinylated antibody using a streptavidin-DNA conjugate, or directly linking DNA to antibodies. However, both methods have a drawback in that a small molecule or DNA has to be linked to the antibody, so as to cause loss of its orientation or chemical modification of antigen-binding site.

### DISCLOSURE

### Technical Problem

It is an object of the present invention to solve the problem that antibodies lose their orientation upon binding to an immunosensor, and to provide techniques for easily immobilizing antibodies on a variety of solid supports in a consistent orientation using well-defined DNA surfaces.

### Technical Solution

Previously, the present inventors have prepared an N-terminal cysteine-tagged protein G variant (Korean Patent Application No. 10-2007-0052560), and confirmed its usefulness through experiments, in order to solve the problem that antibodies lose their orientation upon binding to an immunosensor. Also, based on the invention, the present inventors have prepared a protein G conjugate (gA-G) by chemically linking an oligonucleotide (gA) having an amine group with the cysteine-tagged protein G variant using a linker capable of selectively reacting with both amine and thiol groups. They found that antibodies can be easily immobilized on a variety of solid supports in a consistent orientation and on intended areas of the surfaces by using the protein G conjugate, thereby completing the present invention.

### DESCRIPTION OF DRAWINGS

FIG. 1 shows binding domains (B1 and B2) of Streptococcal protein G that binds with antibodies,

FIG. 2 shows the structure of protein G variant used in the present invention and an amino acid sequence of B2, which is one of domains binding with antibodies,

FIG. 3 is a photograph of protein electrophoresis (SDS-PAGE) showing the expression patterns of the cysteine-tagged protein G variants in E. coli transformed with the expression vector shown in FIG. 2,

FIG. 4 is a diagram showing a biosensor or biochip, prepared by immobilizing the protein G conjugate (gA-G) having an oligonucleotide (gA) on the surface of gold thin film having the complementary oligonucleotide (cA), and then immobilizing an antibody,

FIG. 5 is a photograph of protein electrophoresis (SDS-PAGE) to analyze the protein G conjugate (gA-G),

FIG. 6 is a graph showing the changes in the surface plasmon resonance signal to measure the reaction of the protein G conjugate (gA-G), complementary oligonucleotide (gA), and noncomplementary control oligonucleotide (gB) with the oligonucleotide (cA) on the surface of gold thin film,

FIG. 7 is a graph showing the changes in the surface plasmon resonance signal to detect the reaction of 100 nM PSA and its antibody in the protein G conjugate (gA-G)-immobilized biosensor,

FIG. 8 is a photograph obtained by a fluorescent scanner, in which after linking the oligonucleotide (cA) to the epoxy group on the glass surface, an array was fabricated to immobilize the oligonucleotides (cA or cB) using a DNA arrayer, and then the surface was treated with the protein G conjugate (gA-G) and Cy3-mouse IgG1 (1 nM) labeled with a fluorescent marker Cy3, and

FIG. 9 is a photograph of agarose gel electrophoresis to analyze the formation of antibody-immobilized gold nano-particle, in which (A) is a photograph of agarose gel after reacting the gold nano-particle linked with oligonucleotide (cA) (AuNP-cA) with the complementary oligonucleotide (gA), protein G conjugate (gA-G), noncomplementary control oligonucleotide (gB), and noncomplementary oligonucleotide (gB)-protein G variant (gB-G), (B) is a photograph of agarose gel for analysis of antibody immobilization, after reacting the protein G conjugate (gA-G) with the gold nano-particles having two different numbers of oligonucleotide (cA) (AuNP-cA-I, AuNP-cA-II) and removing the unreacted protein G conjugate (gA-G), and (C) is a schematic diagram showing the IgG labeled AuNP-cA-I and AuNP-cA-II.

### BEST MODE

It is an object of the invention to provide a protein G conjugate (gA-G), which is prepared by linking an N-terminal cysteine-tagged protein G variant with an oligonucleotide (gA) comprising an amine group using a linker capable of selectively reacting with both amine and thiol groups.

It is another object of the invention to provide a method for preparing the protein G conjugate (gA-G conjugate), comprising the step of chemically linking the protein G variant with an oligonucleotide (gA) comprising an amine group using a linker capable of selectively reacting with both amine and thiol groups.

It is still another object of the invention to provide a biosensor fabricated by adhering the protein G conjugate (gA-G conjugate) onto the surface of a solid support, and a method for fabricating a biochip and a biosensor, characterized in that the protein G conjugate is linked to the solid support, the surface of which is linked with an oligonucleotide (cA) having a DNA sequence complementary to the oligonucleotide (gA) comprising an amine group.

It is still another object of the invention to provide a method for analyzing an antigen using the biochip or biosensor.

In one embodiment to achieve the object of the present invention, the present invention relates to a protein G conjugate (gA-G conjugate), which is prepared by linking an N-terminal cysteine-tagged protein G variant with an oligonucleotide (gA) comprising an amine group using a linker capable of selectively reacting with both amine and thiol groups.

The N-terminal cysteine-tagged protein G variant used in the present invention has the following structure.

Aₓ-Cys-L_{y}-Protein G-Q_{z}

(wherein A is an amino acid linker, L is a linker linking a protein G with a cysteine tag, Q is a tag for protein purification, x is 0 to 2, and y or z is 0 or 1, respectively)

Protein G is a bacterial cell wall protein isolated from the group G streptococci, and has been known to bind to Fc and Fab regions of a mammalian antibody (J. Immunol. Methods 1988, 112, 113-120). However, the protein G has been known to bind to the Fc region with an affinity about 10 times greater than the Fab region. A DNA sequence of native protein G was analyzed and has been disclosed. A Streptococcal protein G and Staphylococcal protein A are among various proteins related to cell surface interactions, which are found in Gram-positive bacteria, and have the property of binding to an immunoglobulin antibody. The Streptococcal protein G variant, inter alia, is more useful than the Staphylococcal protein A, since the Streptococcal protein G variant can bind to a wider range of mammalian antibodies, so as to be used as a suitable receptor for the antibodies.

The origin of the protein G used in the present invention is not particularly limited, and the native protein G, an amino acid sequence of which is modified by deletion, addition, substitution or the like, may be suitably used for the purpose of the present invention, as long as it holds the ability to bind to an antibody. In one embodiment of the present invention, only the antibody-binding domains (B1, B2) of the Streptococcal protein G were used.

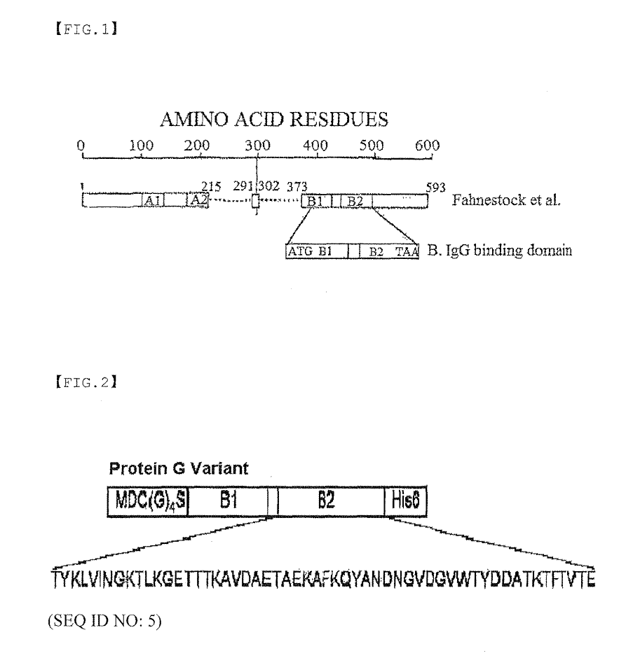

The protein G-B1 domain consists of three β-sheets and one α-helix, and the third β-sheet and α-helix in its C-terminal part are involved in binding to the antibody Fc region. The B1 domain is represented by SEQ ID NO. 1, and the B2 domain is represented by SEQ ID NO. 2. As the amino acid sequences of B1 and B2 domains are compared to each other, there are differences in the four sequences, but little difference in their structures. In one embodiment of the present invention, a B1 domain, in which ten amino acids were deleted at its N-terminus, was used (FIG. 1). It was reported that even though a form of the B1 domain having a deletion of ten amino acid residues from its N-terminus was used, there was no impact on the function of binding with an antibody (Biochem. J. (1990) 267, 171-177, J. MoI. Biol (1994) 243, 906-918, Biochemistry (2000) 39, 6564-6571).

As used herein, the term “cysteine tag (Cys)” refers to a cysteine, which is fused at the N-terminus of protein G. A preferred cysteine tag consists of one cysteine.

In the protein G variant of the present invention, the cysteine tag may be directly linked to the protein G by a covalent bond, or may be linked through a linker (L). The linker is a peptide having any sequence, which is inserted between the protein G and cysteine. The linker may be a peptide consisting of 2 to 10 amino acids. In embodiments of the present invention, the linker consisting of 5 amino acids was used. The cysteine tag of the present invention is not inserted inside the amino acid sequence of the protein G, and it provides the protein G with orientation upon attaching to a solid support. If the linker is attached, a thiol group is readily exposed to the outside. Thus, the protein G can be more efficiently bound to a biosensor with directionality.

In addition, 0 to 2 amino acid (s) may be linked to the cysteine tag of the protein G variant used in the present invention. A preferred amino acid is methionine.

In order to easily isolate the protein G variant of the present invention, a tag (Q) for protein purification may be further included at its C-terminus. In embodiments of the present invention, hexahistidine was tagged at its C-terminus, but as the tag for protein purification, any known tag can be used for the purpose of the invention without limitation. The variant of the present invention may contain methionine, which serves as an initiation codon in prokaryotes, or not. In one embodiment of the present invention, the present inventors prepared a one cysteine-tagged variant.

The protein G variants of the present invention can be prepared by a known method such as a peptide synthesis method, in particular, efficiently prepared by a genetic engineering method. The genetic engineering method is a method for expressing large amounts of the desired protein in a host cell such as E. coli by gene manipulation, and the related techniques are described in detail in disclosed documents (Molecular Biotechnology: Principle and Application of Recombinant DNA; ASM Press: 1994, J. chem. Technol. Biotechnol. 1993, 56, 3-13). Using the known techniques, a nucleic acid sequence encoding the protein G variant used in the present invention is contained in a suitable expression vector, and a suitable host cell is transformed with the expression vector, and cultured to prepare the protein G variants. The preparation method of the protein G variant used in the present invention is described in detail in Korean Patent Application No. 10-2007-0052560, applied by the present inventors, the entire contents of which are fully incorporated herein by reference.

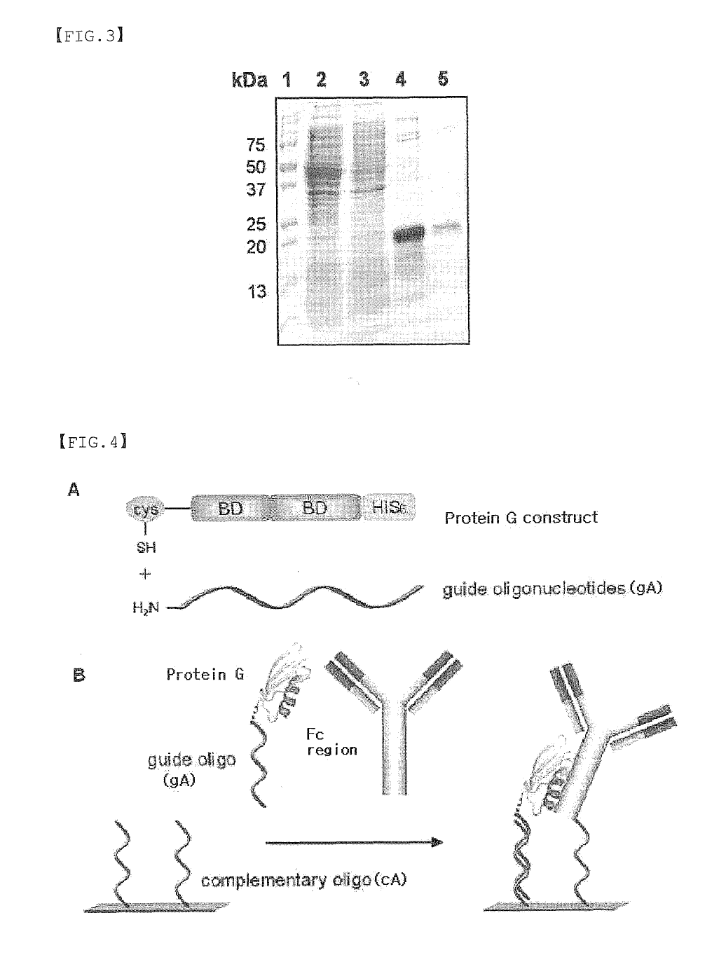

In one embodiment of the present invention, an expression vector (pET-cys1-L-proteinG) comprising a base sequence that encodes the N-terminal cysteine-tagged Streptococcal protein G variant was prepared as shown in FIG. 2.

Cysteine is an amino acid having a thiol group, and has been known to specifically immobilize a protein by its insertion into the protein (FEBS Lett. 1990, 270, 41-44, Biotechnol. Lett. 1993, 15, 29-34). Disclosed is a method for binding cysteine at the C-terminus of Streptococcal protein G. However, in the present invention, cysteine having a thiol group was used to tag the N-terminus, which is remote from the active domain of the Streptococcal protein G variant. The active domain of the Streptococcal protein G that binds with an antibody is located in its C-terminus (the third β-sheet and α-helix). Accordingly, cysteine was not used to tag the inside of the protein G variant but at the N-terminus thereof, thereby minimizing the loss of antibody-binding ability, in which the loss can occur by tagging the C-terminus with cysteine residues.

In embodiments of the present invention, the cysteine-tagged Streptococcal protein G variant was prepared (Example 1). As mentioned above, after gene manipulation, the gene was inserted into a protein expression vector to express the protein, and then the protein G variant was separated by protein electrophoresis.

As used herein, the oligonucleotide (guide oligonucleotide, hereinafter, also referred to as gA) is an oligomer of 18 to 30 nt in length, and may include DNA, RNA, PNA and LNA, preferably oligo DNA. Any sequence, readily selected by those skilled in the art, may be used depending on the purpose, and may be prepared by a known method or a commercially available sequence, for example, a custom oligonucleotide (manufactured by Bioneer or IDT) may be used. The method of oligomer preparation is well known in the art. In addition, the oligonucleotide (gA) used in the present invention comprises an amine group to bind with the protein G via a linker, and the amine group may be located at the 5′-end, 3′-end or inside of the base sequence. To include the amine group in the oligonucleotide (gA), a specific region of the oligonucleotide (gA) may be modified with the amine group by a known method in the art. A preferred oligonucleotide (gA) is an oligonucleotide modified with the amine group at its 5′-end. The amine group of the oligonucleotide is linked to the protein G variant via a linker.

In addition, the oligonucleotide (gA) used in the present invention has a base sequence being complementary to an oligonucleotide (hereinafter, referred to as cA), which is linked onto the surface of the biosensor.

In the present invention, a linker (C) capable of reacting with both amine and thiol groups is used to prepare the protein G conjugate by linking the oligonucleotide (gA) with the protein G variant. The linker of the present invention is used for the purpose of linking the oligonucleotide (gA) comprising an amine group with the protein G variant, and exemplified by Sulfo-SMCC (Sulfosuccinimidyl 4-(N-maleimidomethyl)cyclohexane-1-carboxylate), BMPS (N-[Maleimidopropyloxy]succinimide ester), GMBS (N-[Malwimidobutyryloxy]succinimide ester) and SMPB (Succinimidyl 4-[p-maleimidophenyl]butyrate), but any linker may be used without limitation, as long as it has a property of selectively reacting with both amine and thiol groups. A preferred linker is Sulfo-SMCC.

The oligonucleotide (gA) modified with an amine group at its end and the protein G variant are linked to each other via the linker (C) to prepare the protein G conjugate (gA-G). In this connection, the protein G variant and oligonucleotide (gA) of the present invention have one thiol group and one amine group, respectively. Thus, upon forming the conjugate, the oligonucleotide (gA) and protein G variant are linked to each other one by one.

The protein G conjugate (gA-G) according to the present invention binds in a directional manner with oligonucleotide (cA) on the surface of the solid support of a biosensor by complementary binding, thereby efficiently binding with antibodies. Thus, the protein G conjugate can be satisfactorily used in biochips and biosensors which utilize antigen-antibody reactions.

In still another embodiment, the present invention relates to a method for preparing the protein G conjugate (gA-G), comprising the step of linking the protein G variant and the oligonucleotide (gA) modified with an amine group at its end to a linker capable of reacting with both amine and thiol groups by a covalent bond.

The method for preparing the protein G conjugate (gA-G) according to the present invention, as mentioned above, comprises the step of linking the protein G variant and the oligonucleotide (gA) modified with an amine group at its end to a linker capable of reacting with both amine and thiol groups by a covalent bond, in which any one of protein G variant and oligonucleotide (gA) may be first linked to the linker, and then the other one may be linked thereto.

In one preferred embodiment, the preparation method of the present invention may further include the step of isolating and purifying the protein G conjugate (gA-G) after the conjugate formation. In the isolation/purification step, one or more known methods for isolating/purifying a protein may be suitably selected by those skilled in the art.

In a specific embodiment, the present inventors isolated the protein G conjugate (gA-G), which is prepared by linking the oligonucleotide (gA) modified with an amine group at its 5′-end and the Streptococcal protein G variant tagged with one cysteine to Sulfo-SMCC, by chromatography using both of the column packed with anion exchange excellulose and the column packed with IDA excellulose.

In still another embodiment, the present invention relates to a biochip or a biosensor fabricated by linking the protein G conjugate (gA-G) onto the surface of a solid support, and to a method for fabricating a biochip or a biosensor, comprising the steps of

a) linking an oligonucleotide (cA), which has a base sequence being complementary to an oligonucleotide (gA) of protein G conjugate (gA-G), on the surface of a solid support,

b) linking the oligonucleotide (cA) on the surface of a solid support with the oligonucleotide (gA) of the protein G conjugate (gA-G); and

c) linking an antibody with the protein G conjugate (gA-G) immobilized on the solid support.

Examples of the solid support include metal or membrane, ceramic, glass, polymer surface or silicone, as described in the following Table 1. A preferred solid support is a gold thin film or gold nano-particle.

TABLE 1
Substrate for self-assembled monolayer formation of protein
having cysteine group

| Presence or | | | |
|---|---|---|---|
| absence of | | Applications | |
| surface chemical | Type of | Thin film | Nano-particle or |
| pretreatment | substrate | surface | nano-structure |
| Absence | Ag | ◯ | |
| | Ags | | ◯ |
| | Au | ◯ | ◯ |
| | CdSe | | ◯ |
| | CdS | | ◯ |
| | AuAg | | ◯ |
| | AuCu | | ◯ |
| | Cu | ◯ | ◯ |
| | FePt | | ◯ |
| | GaAs | ◯ | |
| | Ge | ◯ | |
| | Hg | ◯ | |
| | Pd | ◯ | ◯ |
| | Pt | ◯ | ◯ |
| | Stainless | ◯ | |
| | Steel316L | | |
| | Zn | ◯ | |
| | ZnSe | ◯ | |
| | PdAg | | ◯ |
| | Ru, Ir | | ◯ |
| Presence (maleimide | Membrane | ◯ | |
| group, epoxy group, | Ceramic | ◯ | |
| nitrophenol proline | Glass | ◯ | |
| group, and methyl | Polymer | ◯ | |
| iodide group) | surface | | |
| | Silicone | ◯ | |

In addition, on the surface of the solid support, the oligonucleotide (complementary oligonucleotide, hereinafter also referred to as cA) having a base sequence being complementary to the oligonucleotide (gA) of the protein G conjugate (gA-G) of the present invention is linked. The oligonucleotide may be linked onto the surface of the solid support by a known method which is selected by those skilled in the art, depending on the structure of the solid support of a biochip and biosensor. For example, in the case of a glass slide, the complementary oligonucleotide (cA) modified with an amine group may be linked onto the glass slide activated with an epoxy group, and in the case of a gold surface, the complementary oligo DNA (cA) modified with a thiol group may be linked thereto, but is not limited thereto.

In the biochip and biosensor of the present invention, the oligonucleotide (gA) which constitutes the protein G conjugate (gA-G) of the present invention is linked onto the solid support by complementary binding with the oligonucleotide (cA) on the surface of the solid support, and the protein G conjugate linked to the solid support binds with an antibody. The biochip and biosensor of the present invention may be easily fabricated by contacting the protein G conjugate and antibody with the solid support.

In still another embodiment, the present invention relates to a method for analyzing an antigen using the biochip or biosensor.

The biochip or biosensor of the present invention is one type of immunosensors, and thus antigen analysis may be performed by any method using the immunosensor, which is widely known in the art. A surface plasmon resonance-based method may be preferably used to analyze the antigen.

Hereinafter, the present invention will be described in detail with reference to Examples. However, these Examples are for illustrative purposes only, and the invention is not intended to be limited thereto.

### MODE FOR INVENTION

### Example 1

### Protein Expression Analysis of Cysteine-Tagged Streptococcal Protein G Variant

<1-1> Gene Preparation of Cysteine-Tagged Streptococcal Protein G Variant

Two primers were prepared in order to tag with cysteine at the N-terminus. In the base sequence of the 5′-primer, an initiation codon (ATG) was followed by GAT (Asp codon) and TGC (cysteine codon), and in order to provide a link to protein G, GGC GGC GGC GGC AGC (four Gly codons and one Ser codon) were included. Furthermore, in order to insert the gene into an expression vector pET21a (Novagen), the NdeI restriction site was introduced into the N-terminal primer and the XhoI restriction site was introduced into the C-terminal primer. The Streptococcal genomic gene was obtained, and a polymerase chain reaction (PCR) was performed with the primers. Thus, only the amino acid regions (B1 [a form having 10 initial amino acid residues cleaved], B2), which are known as domains to which an antibody binds, were obtained. The obtained fragments were cleaved with the restriction enzymes, which were the same enzymes as introduced into each primer. Then, the cleaved fragment was inserted into the pET21a vector cleaved with NdeI and XhoI restriction enzymes to prepare a pET-cys1-L-protein G vector. The expression vector expresses Met at the N-terminus.

5′ Primer 1: Sense

(SEQ ID NO. 1)

5-GGGAATTCCATATGCATTGCGGCGGCGGCGGCAGCAAAGGCCAAACAA
CTACTGAAGCT-3
3′ Primer 2: Antisense

(SEQ ID NO. 2)

5-GAGCTCGAGTTCAGTTACCGTAAAGGTCTTAGTC-3

<1-2> Protein Electrophoresis of Cysteine-Tagged Streptococcal Protein G Variants

E. coli BL21 cells were transformed with the prepared pET-cys1-L-protein G, and cultured with shaking at 37° C. until an O.D. (optical density, A600 nm) became 0.6. Then, IPTG (isopropyl β-D-thiogalactopyranoside, total concentration of 1 mM) was added thereto, so as to induce protein expression at 25° C. After 14 hrs, the E. coli cells were centrifuged, and the obtained cell pellets were disrupted by sonication (Branson, Sonifier 450, 3 KHz, 3 W, 5 min) to give a total protein solution. The total protein solution was separated by centrifugation into a solution of soluble protein fraction and a solution of non-soluble protein fraction. To purify the protein solution, a solution of disrupted cells in which the recombinant gene conjugated with hexahistidine were expressed, was loaded on a column packed with IDA excellulose. The recombinant protein conjugated with histidine was eluted with an eluent (50 mM Tris-Cl, 0.5 M imidazole, 0.5 M NaCl, pH 8.0). To purify the obtained protein solution once more, the solution was loaded on a column packed with Q cellulose, and eluted with 1 M NaCl. Then, the eluted protein solution was dialyzed in PBS (phosphate-buffered saline, pH 7.4) buffer solution.

For protein electrophoresis, the protein solution obtained in the above was mixed with a buffer solution (12 mM Tris-Cl, pH 6.8, 5% glycerol, 2.88 mM mercaptoethanol, 0.4% SDS, 0.02% Bromophenol Blue) and heated at 100° C. for 5 min, and then the resultant was loaded on a poly-acrylamide gel, which consisted of a 1 mm-thick 15% separating gel (pH 8.8, width 20 cm, length 10 cm) covered by a 5% stacking gel (pH 6.8, width 10 cm, length 12.0 cm). Subsequently, electrophoresis was performed at 200 to 100 V and 25 mA for 1 hr, and the gel was stained with a Coomassie staining solution to confirm the recombinant protein.

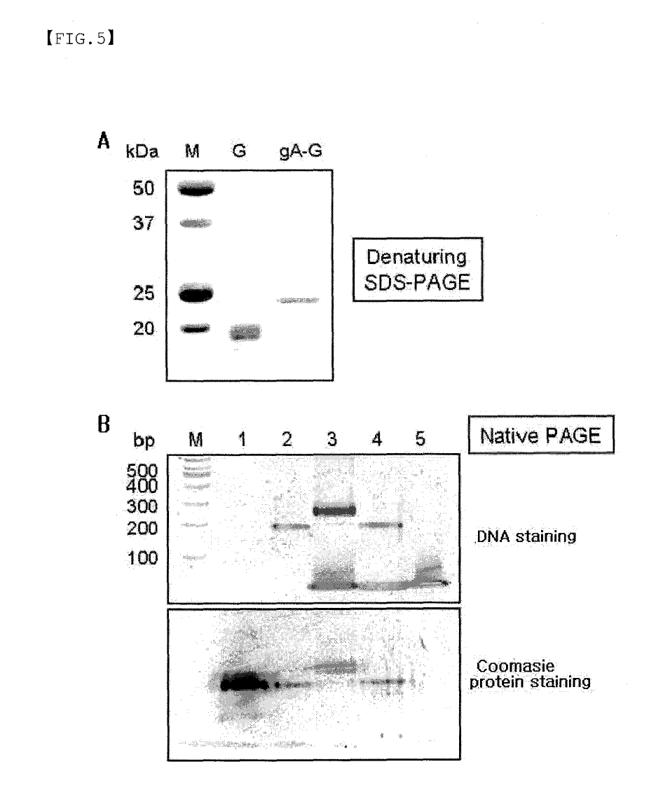

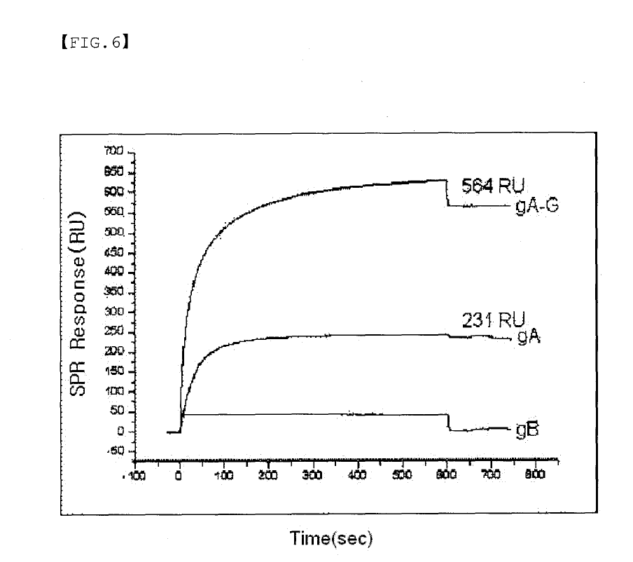

The description of lanes in FIG. 3 is as follows;

Lane 1: protein size marker,

Lane 2: total protein of E. coli transformed with plasmid pET-cys1-L-proteinG,

Lane 3: soluble protein fraction of E. coli transformed with plasmid pET-cys1-L-protein G,

Lane 4: purified protein by IDA column,

Lane 5: purified protein by Q cellulose column.

### Example 2

### Preparation of Protein G Conjugate (gA-G)

Using Sulfo-SMCC (Sulfosuccinimidyl 4-(N-maleimidomethyl)cyclohexane-1 carboxylate), an oligonucleotide (gA) modified with an amine group and a Streptococcal protein G variant tagged with one cysteine were chemically linked to each other to prepare a protein G conjugate (gA-G).

In particular, 60 nmol of the oligo DNA (gA) modified with an amine group at 5′-end was dissolved in 400 μl of 0.25 M phosphate buffer, and then reacted with 1.5 mg of Sulfo-SMCC (3400 nmol) dissolved in 75 μl of DMF solution. The mixture was reacted at normal temperature for 1 hr, and then the activated oligo DNA (gA) was separated from the excess unreacted Sulfo-SMCC using a binding buffer (20 mM Tris, 50 mM NaCl, 1 mM EDTA pH7.0) by Sephadex G25 gel filtration. While performing the activation of oligo DNA, the cysteine tagged-protein G variant was reacted with 20 mM DTT for complete reduction, followed by gel filtration to remove DTT. Consequently, the obtained cysteine tagged-protein G variant was immediately reacted with the activated oligo DNA (gA) at normal temperature for 2 hrs.

The residual oligo DNA (gA) which was not linked to the protein G was separated from the protein G variant and cysteine tagged-protein G conjugate (gA-G) using a His-tagged affinity column (IDA column). Then, the protein G conjugate (gA-G) was purified using an ion exchange column to remove the unbound protein G variant.

The protein G conjugate (gA-G) was separated by chromatography with two columns (column packed with IDA excellulose, and column packed with anion exchange Q cellulose), and then the protein G conjugate (gA-G) was analyzed by protein electrophoresis (Native gel, SDS-PAGE). After protein electrophoresis, the gels were stained with Gel Red and Coomassie, which are DNA and protein-specific staining reagents, respectively. As a result, it was found in a Native gel that the protein G variant-DNA conjugate (gA-G) was specifically linked to the oligomer (cA) having a complementary DNA sequence to cause a difference in its migration (lane 2 vs lane 3). Also, the band strength was found to be increased only in the DNA staining. Therefore, it can be seen that the protein G conjugate (gA-G) was specifically reacted with the complementary oligomer (cA).

The above results indicate that the protein G variant (G) and the oligomer (gA) are linked to each other one-to-one in the prepared protein G conjugate (gA-G) (FIG. 5).

### Example 3

### Fabrication of Protein G Conjugate (gA-G)-Immobilized Biosensor and Biochip

The oligo DNA (gA) was chemically linked to the one cysteine-tagged Streptococcal protein G variant, and then reacted with the surface of gold thin film, on which the oligo DNA (cA) complementary to oligo DNA (gA) was linked, to fabricate a protein G conjugate (gA-G)-immobilized biosensor and biochip.

In particular, the oligo DNA (cA) was reacted with the surface of gold thin film, and then changes in the surface plasmon resonance signal were measured by means of a surface plasmon resonance (SPR)-based biosensor to detect the immobilization reaction of the complementary oligo DNA (gA), protein G conjugate (gA-G), and noncomplementary control oligo DNA (gB) in real-time.

As a result, when the noncomplementary control oligo DNA (gB) was injected, there was little change in the surface plasmon resonance signal. When the complementary oligo DNA (gA, 7.5 kDa) was injected, the surface plasmon resonance signal was increased by 231 RU. When the oligo DNA (gA)-protein G conjugate (gA-G, 21.5 kDa) was injected, the surface plasmon resonance signal was increased by 564 RU, indicating that the oligo DNA (gA, 7.5 kDa) and protein G conjugate (gA-G, 21.5 kDa) were specifically linked onto the surface of oligo DNA (cA)-immobilized gold thin film.

In addition, the numbers of oligo DNA (gA, 7.5 kDa) and protein G conjugate (gA-G, 21.5 kDa) linked on the surface (mm²) were calculated. The number of oligo DNA (gA, 7.5 kDa) was 1.8×10¹⁰ molecules/mm². The number of protein G conjugate (gA-G, 21.5 kDa) was 1.6×10¹⁰ molecules/mm², which had a slightly lower density than the oligo DNA (gA, 7.5 kDa). The result indicates that the protein G variant slightly interfered with the complementary reaction of oligo DNA.

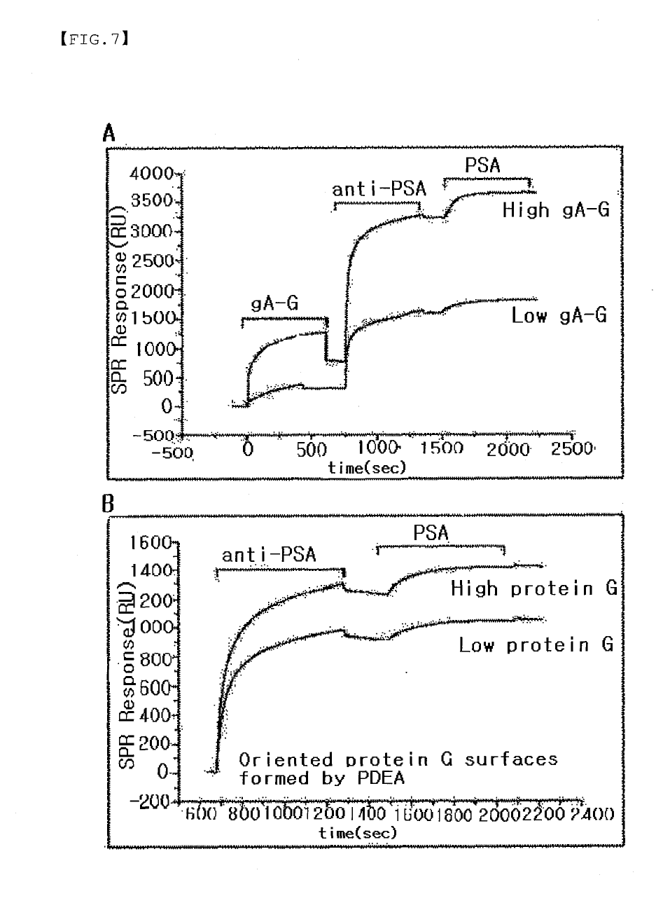

However, changes in the surface plasmon resonance signal were measured by means of a surface plasmon resonance (SPR)-based biosensor to detect the ability of the protein G conjugate (gA-G) to efficiently bind with an antibody, upon reacting the surface with various antibodies (FIG. 6).

### Example 4

### Detection of Antigen Using Protein G Conjugate (gA-G)-Immobilized Biosensor

Antigen detection was performed using the biosensor which binds with an antibody via the Streptococcal protein G conjugate immobilized by complementary reaction of oligo DNA.

In particular, 50 nM protein G conjugate (gA-G) was immobilized on the surface of complementary oligo DNA (cA)-immobilized gold thin film for the immobilization time of 10 min and 7 min, and then changes in the surface plasmon resonance signal were measured by means of a surface plasmon resonance-based biosensor to detect the reaction between an antibody (anti-human Kallikrein 3/PSA antibody, R&D systems, 100 nM) and its antigen (Recombinant Human kallikrein 3/PSA, 100 nM).

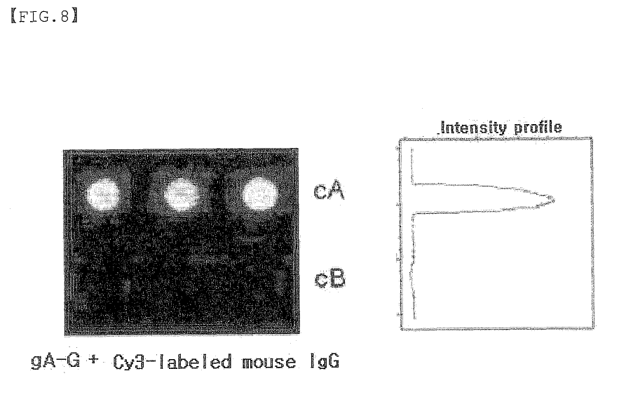

As a result, when the protein G conjugate (gA-G) was reacted for 10 min, the surface plasmon resonance signal was increased by 775 RU. When the protein G conjugate (gA-G) was reacted for 7 min, the surface plasmon resonance signal was increased by 297 RU. When the antibody was reacted with the gA-G immobilized surface of 775 RU, the surface plasmon resonance signal was increased by 2440 RU. When the antibody was reacted with the gA-G immobilized surface of 297 RU, the surface plasmon resonance signal was increased by 1296 RU. When the antigen was reacted with the antibody of 2440 RU on the gA-G immobilized surface of 775 RU, the surface plasmon resonance signal was increased by 435 RU. When the antigen was reacted with the antibody of 1296 RU on the gA-G immobilized surface of 297 RU, the surface plasmon resonance signal was increased by 231 RU (FIG. 7).

### Example 5

### Antibody Immobilization Using Protein G Conjugate Linked onto DNA Array

The DNA array was fabricated on other surfaces than the surface of gold thin film, and then antibody was immobilized using the protein G conjugate linked to DNA (gA, 21.5 kDa).

In particular, the oligonucleotides (cA and cB) with amine groups were linked to the epoxy groups on the glass surface, an array was fabricated using a DNA arrayer, and then non-specific reaction was blocked with BSA. Then, a mixed solution of the protein G conjugate (gA-G) and antibody labeled with a fluorescent marker (Monoclonal mouse IgG-Cy3 (150 nM)) were reacted with the surface, and fluorescent signals were measured using a fluorescent scanner.

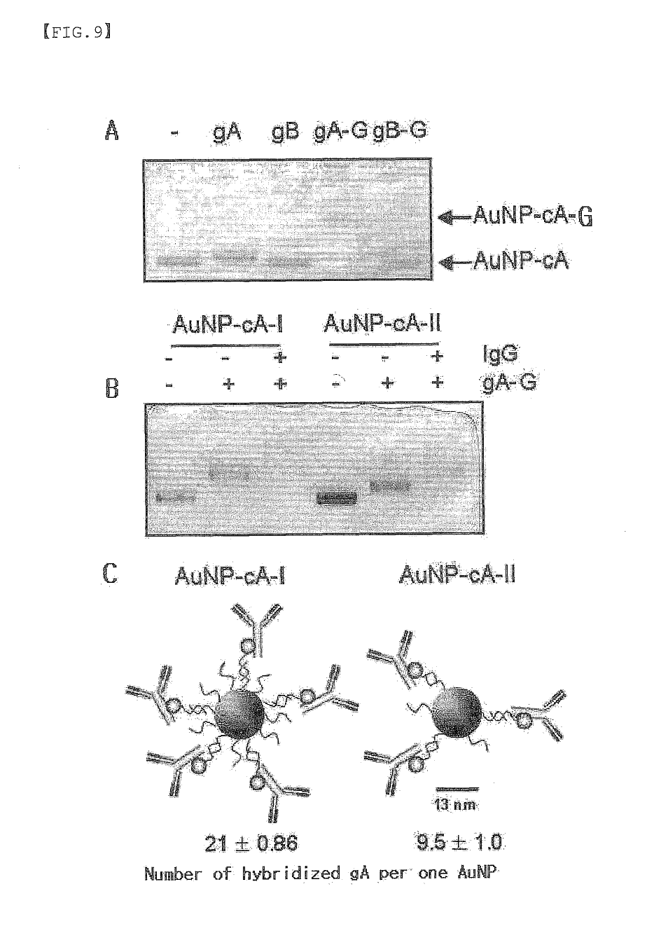

As a result, since the protein G conjugate (gA-G) binding with the antibody binds with the complementary oligonucleotide cA, fluorescence was observed not in the oligonucleotide cB but in the complementary oligonucleotide cA, indicating that the antibody can be easily immobilized using a DNA array without non-specific reaction (FIG. 8).

### Example 6

### Fabrication of Antibody-Immobilized Gold Nano-Particle Via Protein G Conjugate (gA-G)

Antibody-immobilized gold nano-particles were fabricated using the protein G conjugate (gA-G).

In particular, when the complementary oligonucleotide cA-linked gold nano-particle was linked to gA-G (21.5 KDa) and gA (7.5 KDa), the gA-G (21.5 KDa)-linked band, which is a relatively upper band, was less migrated than the gA (7.5 KDa)-linked band in the negative gel. In addition, to sufficiently link the protein G conjugate (gA-G) to cA, two different numbers of complementary oligonucleotide (cA) were linked to the gold nano-particles (allowed to link with the average number of 21 or 9.5 gA), the protein G conjugate (gA-G) was linked thereto, and then antibodies were linked (human IgGs).

As a result, it was found that more numbers of protein G conjugate (gA-G) and antibody were linked onto the gold nano-particle capable of binding with the average number of 21 gA.

In the present experiment, the gold nano-particle-cA linked with gA-G (21.5 KDa) was recovered using a centrifuge, and then any unreacted antibody was removed using the hexahistidine tagged to the protein variant. Based on the above results, the protein G conjugate (gA-G) is very useful for immobilizing antibodies on the gold nano-particle (FIG. 9).

### INDUSTRIAL APPLICABILITY

The protein G conjugate (gA-G) according to the present invention, which is prepared by linking an N-terminal cysteine-tagged protein G variant with an oligonucleotide via a linker, binds in a directional manner with oligonucleotide (cA) on the surface of the solid support of a biosensor, and thus efficiently binds with antibodies, thereby being satisfactorily used in biochips and biosensors which utilize antigen-antibody reactions.

1. A protein G conjugate (gA-G conjugate) which is prepared by linking an N-terminal cysteine-tagged protein G variant with an oligonucleotide (gA) comprising an amine group using a linker capable of selectively reacting with both amine and thiol groups, represented by the following Formula:
Aₓ-Cys-L_{y}-Protein G-Q_{z}
(wherein A is an amino acid linker, L is a linker linking a protein G with a cysteine tag, Q is a tag for protein purification, x is 0 to 2, and y or z is 0 or 1, respectively).

2. The protein G conjugate according to claim 1, wherein the oligonucleotide (gA) is selected from the group consisting of oligo DNA, RNA, PNA (peptide nucleic acid) and LNA (locked nucleic acid) and has a length of 18 to 30 nt.

3. The protein G conjugate according to claim 1, wherein the oligonucleotide (gA) comprising an amine group is modified with an amine group at its 5′-end.

4. The protein G conjugate according to claim 1, wherein the linker capable of reacting with both amine and thiol groups is selected from the group consisting of Sulfo-SMCC (Sulfosuccinimidyl 4-(N-maleimidomethyl)cyclohexane-1-carboxylate), BMPS (N-[Maleimidopropyloxy]succinimide ester), GMBS (N-[Malwimidobutyryloxy]succinimide ester), and SMPB (Succinimidyl 4-[p-maleimidophenyl]butyrate).

5. The protein G conjugate according to claim 1, wherein the protein G variant and oligonucleotide (gA) are linked to each other one by one.

6. The protein G conjugate according to claim 1, wherein the linker (L) linking a protein G with a cysteine tag is a peptide consisting of 2 to 10 amino acids, preferably an amino acid sequence of DDDDK (Asp-Asp-Asp-Asp-Lys) (SEQ ID NO:4).

7. A method for preparing the protein G conjugate of claim 1 comprising the step of linking an N-terminal cysteine-tagged protein G variant and an oligonucleotide (gA) comprising an amine group with a linker capable of reacting with both amine and thiol groups by a covalent bond, represented by the following Formula:
Aₓ-Cys-L_{y}-Protein G-Q_{z}
(wherein A is an amino acid linker, L is a linker linking a protein G with a cysteine tag, Q is a tag for protein purification, x is 0 to 2, and y or z is 0 or 1, respectively).

8. The method for preparing the protein G conjugate according to claim 7, further comprising the step of isolating and purifying the protein G conjugate after the conjugate formation.

9. A biochip which is fabricated by linking the protein G conjugate of claim 1 onto the surface of a solid support.

10. The biochip according to claim 9, wherein an oligonucleotide (cA) having a base sequence complementary to the oligonucleotide (gA) of the protein G conjugate is linked onto the surface of the solid support, wherein the solid support is selected from the group consisting of ceramic, glass, polymer, silicone and metal, and wherein the biochip is a gold thin film or gold nano-particle.

11. The biochip according to claim 9, wherein an antibody is linked to the protein G conjugate.

12. A method for fabricating a biochip or a biosensor, comprising the steps of
a) linking an oligonucleotide (cA), which has a base sequence being complementary to an oligonucleotide (gA) of the protein G conjugate of claim 1, onto the surface of a solid support;
b) linking the oligonucleotide (cA) on the surface of the solid support with the oligonucleotide (gA) of the protein G conjugate; and
c) linking an antibody with the protein G conjugate immobilized on the solid support.

13. A method for analyzing an antigen using the biochip of claim 9.

14. A biosensor which is fabricated by linking the protein G conjugate of claim 1 onto the surface of a solid support.

15. The biosensor according to claim 14, wherein an oligonucleotide (cA) having a base sequence complementary to the oligonucleotide (gA) of the protein G conjugate is linked onto the surface of the solid support, wherein the solid support is selected from the group consisting of ceramic, glass, polymer, silicone and metal, and wherein the biosensor is a gold thin film or gold nano-particle.

16. The biosensor according to claim 14, wherein an antibody is linked to the protein G conjugate.

17. A method for analyzing an antigen using the biosensor of claim 14.

18-20. (canceled)
